(19) United States
(12) Patent Application Publication
(10) Pub. No.: US 2026/0073784 A1
(43) Pub. Date: Mar. 12, 2026

(54) SYSTEM

(71) Applicant: SoftBank Group Corp., Tokyo (JP)

(72) Inventor: Mikio YAGI, Tokyo (JP)

(21) Appl. No.: 19/314,834

(22) Filed: Aug. 29, 2025

(30) Foreign Application Priority Data
Sep. 10, 2024 (JP) 2024-156057

(51) Int. Cl. G08B 21/04; A61B 5/00; A61B 5/16; G06F 3/01

(52) U.S. Cl. CPC G08B 21/0453; A61B 5/0077; A61B 5/163; A61B 5/165; A61B 5/6803; A61B 5/7278; A61B 5/7282; A61B 5/741; A61B 5/747; G06F 3/012; G06F 3/013; A61B 2503/08; G06F 2203/011

(57) ABSTRACT

The system according to the embodiment comprises a recognition unit, an analysis unit, a provision unit, a detection unit, and a notification unit. The recognition unit analyzes eye movements and recognizes objects being viewed by the elderly person. The analysis unit analyzes information recognized by the recognition unit and generates voice guidance. The provision unit provides the voice guidance generated by the analysis unit. The detection unit detects abnormal movements or conditions of the elderly person using IoT sensor technology. The notification unit notifies designated contacts or medical institutions of abnormalities detected by the detection unit.

### CROSS-REFERENCE TO RELATED APPLICATION

The present application claims priority to and incorporates by reference the entire contents of Japanese Patent Application No. 2024-156057 filed in Japan on Sep. 10, 2024.

### BACKGROUND OF THE INVENTION

### Field of the Invention

The technology of this disclosure relates to the system.

### Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to simultaneously ensure both safety and convenience in the daily lives of elderly people.

### SUMMARY OF THE INVENTION

The system according to the embodiment comprises a recognition unit, an analysis unit, a provision unit, a detection unit, and a notification unit. The recognition unit analyzes eye movements and recognizes objects being viewed by the elderly person. The analysis unit analyzes information recognized by the recognition unit and generates voice guidance. The provision unit provides the voice guidance generated by the analysis unit. The detection unit detects abnormal movements or conditions of the elderly person using IoT sensor technology. The notification unit notifies designated contacts or medical institutions of abnormalities detected by the detection unit.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor with a sign (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) with a sign is a memory where information is temporarily stored and used as a work memory by the processor.

In the following embodiments, a storage with a sign is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) with a sign is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

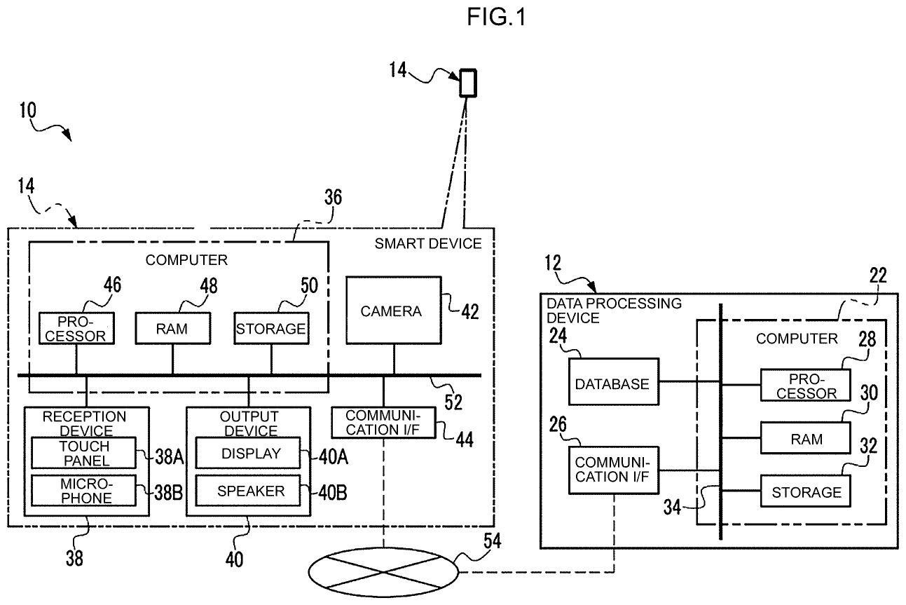

FIG. 1 shows an example configuration of a data processing system 10 according to the first embodiment. As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

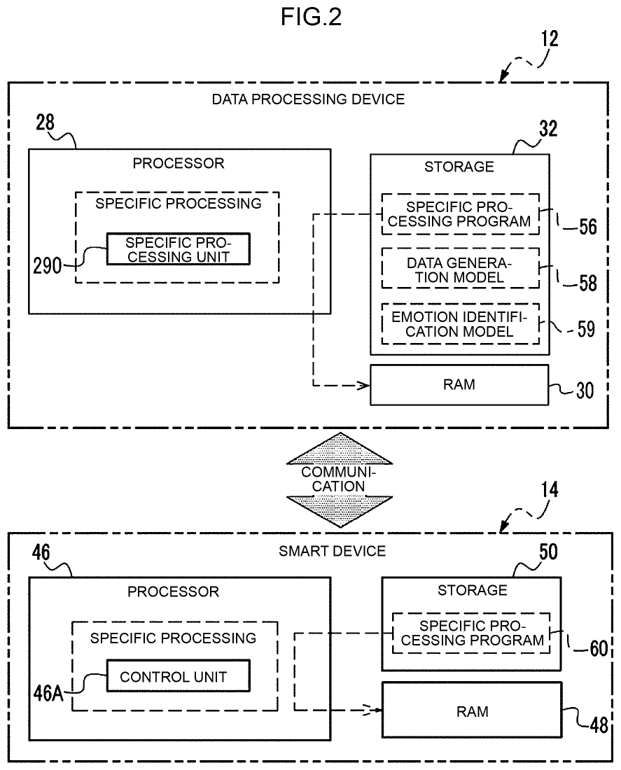

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1 of the Embodiment

The elderly support system according to the embodiment of the present invention is a system that utilizes eye-tracking technology and IoT sensor technology to recognize the daily actions and objects of elderly people and provides various information and advice through voice guidance. This elderly support system recognizes objects and actions being viewed by the elderly person using eye-tracking technology. For example, when a specific medicine or tool is viewed, the system explains its usage or intake method by voice. In addition, IoT sensor technology is introduced to immediately detect abnormal movements or conditions of the elderly person. For example, when a fall or abnormal heart rate is detected, the system has a function to automatically notify designated contacts or medical institutions. This allows elderly people to continue living at home with peace of mind. For example, the system recognizes objects and actions being viewed by the elderly person using eye-tracking technology. At this time, the system analyzes the eye movements in detail and identifies which object is being viewed. For example, when the elderly person looks at a medicine bottle, the system recognizes this information and explains the usage or intake method by voice. This enables the elderly person to use the medicine correctly. Next, IoT sensor technology is introduced to immediately detect abnormal movements or conditions of the elderly person. For example, the system monitors the heart rate and movements of the elderly person in real time using sensors, and immediately notifies when an abnormality is detected. This enables prompt response in emergencies. Furthermore, the system has a function to automatically notify designated contacts or medical institutions. For example, when a fall is detected, a notification is automatically sent to family members or medical institutions. This allows elderly people to continue living safely even when alone. With this mechanism, elderly people can continue living at home with peace of mind. By combining eye-tracking technology and IoT sensor technology, the system ensures the safety of elderly people and supports their daily lives. For example, by explaining the usage of medicine by voice, misuse of medicine is prevented, and by detecting abnormal movements and responding quickly, the risk in emergencies is reduced. Thus, the elderly support system supports the daily lives of elderly people and enables them to continue living at home with peace of mind.

The elderly support system according to the embodiment comprises a recognition unit, an analysis unit, a provision unit, a detection unit, and a notification unit. The recognition unit analyzes eye movements and recognizes objects being viewed by the elderly person. For example, the recognition unit identifies objects being viewed by the elderly person using eye-tracking technology. For example, the recognition unit analyzes the eye movements in detail and identifies which object is being viewed. For example, when the elderly person looks at a medicine bottle, the system recognizes this information and explains the usage or intake method by voice. The analysis unit analyzes information recognized by the recognition unit and generates voice guidance. For example, the analysis unit generates voice guidance based on the information recognized by the recognition unit. For example, the analysis unit uses speech synthesis technology to generate the recognized information as voice guidance. The provision unit provides the voice guidance generated by the analysis unit. For example, the provision unit provides the voice guidance generated by the analysis unit to the elderly person. For example, the provision unit provides voice guidance using a speaker. The detection unit detects abnormal movements or conditions of the elderly person using IoT sensor technology. For example, the detection unit monitors the heart rate and movements of the elderly person in real time using IoT sensor technology and immediately notifies when an abnormality is detected. For example, the detection unit detects falls or abnormal heart rates. The notification unit notifies designated contacts or medical institutions of abnormalities detected by the detection unit. For example, the notification unit automatically notifies designated contacts or medical institutions based on abnormalities detected by the detection unit. For example, when a fall is detected, the notification unit automatically sends a notification to family members or medical institutions. Thus, the elderly support system according to the embodiment supports the daily lives of elderly people and enables them to continue living at home with peace of mind.

The recognition unit can recognize objects being viewed by the elderly person using eye-tracking technology. For example, the recognition unit identifies objects being viewed by the elderly person using eye-tracking technology. For example, the recognition unit analyzes the eye movements in detail and identifies which object is being viewed. For example, when the elderly person looks at a medicine bottle, the system recognizes this information and explains the usage or intake method by voice. By using eye-tracking technology, the recognition unit can accurately recognize objects being viewed by the elderly person. Some or all of the above-described processing in the recognition unit may be performed using AI or may be performed without using AI. For example, the recognition unit can input eye movement data into AI, and the AI can perform object recognition.

The analysis unit can analyze information recognized by the recognition unit and generate voice guidance. For example, the analysis unit generates voice guidance based on the information recognized by the recognition unit. For example, the analysis unit uses speech synthesis technology to generate the recognized information as voice guidance. For example, when the recognition unit recognizes a medicine bottle, the analysis unit generates guidance that explains the usage or intake method by voice. Thus, the analysis unit can analyze recognized information and generate appropriate voice guidance. Some or all of the above-described processing in the analysis unit may be performed using AI or may be performed without using AI. For example, the analysis unit can input information from the recognition unit into AI, and the AI can generate voice guidance.

The provision unit can provide the voice guidance generated by the analysis unit. For example, the provision unit provides the voice guidance generated by the analysis unit to the elderly person. For example, the provision unit provides voice guidance using a speaker. For example, the provision unit provides the voice guidance on the usage or intake method of medicine generated by the analysis unit to the elderly person. Thus, the provision unit can provide the generated voice guidance to the elderly person. Some or all of the above-described processing in the provision unit may be performed using AI or may be performed without using AI. For example, the provision unit can input voice guidance from the analysis unit into AI, and the AI can provide the voice guidance.

The detection unit can detect abnormal movements or conditions of the elderly person using IT sensor technology. For example, the detection unit monitors the heart rate and movements of the elderly person in real time using IoT sensor technology and immediately notifies when an abnormality is detected. For example, the detection unit detects falls or abnormal heart rates. For example, the detection unit monitors the heart rate and movements of the elderly person in real time using sensors and immediately notifies when an abnormality is detected. Thus, by using IoT sensor technology, the detection unit can accurately detect abnormal movements or conditions of the elderly person. Some or all of the above-described processing in the detection unit may be performed using AI or may be performed without using AI. For example, the detection unit can input data from sensors into AI, and the AI can detect abnormalities.

The notification unit can notify designated contacts or medical institutions of abnormalities detected by the detection unit. For example, the notification unit automatically notifies designated contacts or medical institutions based on abnormalities detected by the detection unit. For example, when a fall is detected, the notification unit automatically sends a notification to family members or medical institutions. For example, when the detection unit detects an abnormal heart rate, the notification unit automatically notifies designated contacts or medical institutions. Thus, the notification unit can promptly notify designated contacts or medical institutions of detected abnormalities. Some or all of the above-described processing in the notification unit may be performed using AI or may be performed without using AI. For example, the notification unit can input abnormal data from the detection unit into AI, and the AI can perform the notification.

The recognition unit can improve recognition accuracy by analyzing not only eye movements but also facial expressions and head movements. For example, the recognition unit analyzes facial expressions in conjunction with eye movements to accurately recognize whether a specific object is being viewed. For example, the recognition unit combines head movements and eye movements to more accurately determine the direction of gaze. For example, the recognition unit analyzes changes in facial expressions in real time and performs corrections to improve recognition accuracy. Thus, by analyzing facial expressions and head movements together, recognition accuracy is improved. Some or all of the above-described processing in the recognition unit may be performed using AI or may be performed without using AI. For example, the recognition unit can input facial expression data and head movement data into AI, and the AI can improve recognition accuracy.

The recognition unit can refer to the usage history of an object when recognizing a specific object to correct the recognition result. For example, when the recognition unit recognizes an object that the elderly person has frequently used in the past, it refers to the usage history to correct the recognition result. For example, when the recognition unit recognizes a specific medicine bottle, it performs accurate recognition based on past usage history. For example, the recognition unit refers to the usage history of tools and corrects the recognition result to prevent misrecognition. Thus, by referring to the usage history, the accuracy of recognition results is improved. Some or all of the above-described processing in the recognition unit may be performed using AI or may be performed without using AI. For example, the recognition unit can input usage history data into AI, and the AI can correct the recognition result.

The recognition unit can improve recognition accuracy by using not only the movement of the elderly person's gaze but also voice input. For example, when the elderly person looks at a specific object and says its name by voice, the recognition unit combines gaze and voice to improve recognition accuracy. For example, when the elderly person moves their gaze while giving instructions by voice, the recognition unit integrates gaze and voice for recognition. For example, when the elderly person fixes their gaze while asking a question by voice, the recognition unit combines gaze and voice to correct the recognition result. Thus, by using both gaze movement and voice input, recognition accuracy is improved. Some or all of the above-described processing in the recognition unit may be performed using AI or may be performed without using AI. For example, the recognition unit can input gaze data and voice data into AI, and the AI can improve recognition accuracy.

The recognition unit can adjust recognition accuracy based on environmental information around the elderly person. For example, when the lighting is dim, the recognition unit enhances the analysis of eye movements to improve recognition accuracy. For example, when the surrounding noise is loud, the recognition unit prioritizes gaze analysis to reduce the influence of voice input. For example, when the lighting is bright, the recognition unit combines eye movements and facial expressions to improve recognition accuracy. Thus, by considering environmental information, recognition accuracy is improved. Some or all of the above-described processing in the recognition unit may be performed using AI or may be performed without using AI. For example, the recognition unit can input environmental information data into AI, and the AI can adjust recognition accuracy.

The analysis unit can analyze information from the recognition unit in real time and generate voice guidance immediately. For example, immediately after the recognition unit recognizes a specific object, the analysis unit immediately explains the usage of the object by voice. For example, when the recognition unit recognizes an action of the elderly person, the analysis unit immediately provides appropriate advice by voice. For example, when the recognition unit detects abnormal movements, the analysis unit immediately generates a warning voice. Thus, by analyzing in real time and generating voice guidance immediately, prompt response is possible. Some or all of the above-described processing in the analysis unit may be performed using AI or may be performed without using AI. For example, the analysis unit can input information from the recognition unit into AI, and the AI can generate voice guidance in real time.

The analysis unit can refer to the history of past voice guidance to generate more appropriate guidance. For example, the analysis unit provides optimal guidance based on the history of voice guidance previously received by the elderly person. For example, the analysis unit refers to the history of past voice guidance and adjusts so as not to repeat the same guidance. For example, the analysis unit analyzes the effectiveness of guidance previously received by the elderly person and generates more effective guidance. Thus, by referring to past history, more appropriate voice guidance can be provided. Some or all of the above-described processing in the analysis unit may be performed using AI or may be performed without using AI. For example, the analysis unit can input past voice guidance history data into AI, and the AI can generate guidance.

The analysis unit can generate voice guidance based on the health status of the elderly person. For example, when the heart rate of the elderly person is high, the analysis unit provides advice for relaxation by voice. For example, when the blood pressure of the elderly person is low, the analysis unit provides voice guidance to encourage appropriate actions. For example, the analysis unit monitors the health status of the elderly person in real time and generates appropriate voice guidance. Thus, by considering the health status of the elderly person, more appropriate voice guidance can be provided. Some or all of the above-described processing in the analysis unit may be performed using AI or may be performed without using AI. For example, the analysis unit can input health data of the elderly person into AI, and the AI can generate voice guidance.

The analysis unit can refer to the schedule information of the elderly person to provide voice guidance at an appropriate timing. For example, the analysis unit notifies the medication time by voice based on the schedule of the elderly person. For example, the analysis unit provides advice at an appropriate timing according to the schedule of the elderly person. For example, the analysis unit reminds important appointments by voice based on the schedule information of the elderly person. Thus, by referring to schedule information, voice guidance can be provided at an appropriate timing. Some or all of the above-described processing in the analysis unit may be performed using AI or may be performed without using AI. For example, the analysis unit can input schedule information data into AI, and the AI can determine the timing for providing voice guidance.

The provision unit can provide information not only through voice guidance but also by combining visual guidance. For example, the provision unit displays the procedure for use on a display simultaneously with voice guidance. For example, the provision unit displays visual icons or images together with voice guidance. For example, the provision unit displays the content of the voice guidance as text on a display for visual confirmation. Thus, by combining visual guidance, understanding of information is deepened. Some or all of the above-described processing in the provision unit may be performed using AI or may be performed without using AI. For example, the provision unit can input data of voice guidance and visual guidance into AI, and the AI can provide the information.

The provision unit can adjust the volume and frequency of the voice guidance in consideration of the hearing condition of the elderly person. For example, the provision unit automatically adjusts the volume of the voice guidance according to the hearing condition of the elderly person. For example, the provision unit adjusts the frequency of the voice guidance based on the hearing condition of the elderly person. For example, the provision unit monitors the hearing condition of the elderly person in real time and provides voice guidance at an appropriate volume and frequency. Thus, by adjusting the volume and frequency of the voice guidance according to the hearing condition of the elderly person, more appropriate guidance can be provided. Some or all of the above-described processing in the provision unit may be performed using AI or may be performed without using AI. For example, the provision unit can input hearing data of the elderly person into AI, and the AI can adjust the volume and frequency of the voice guidance.

The provision unit can detect environmental sounds around the elderly person and automatically adjust the volume of the voice guidance. For example, when the surrounding environmental sound is loud, the provision unit automatically increases the volume of the voice guidance. For example, when the surroundings are quiet, the provision unit automatically lowers the volume of the voice guidance. For example, the provision unit adjusts the volume of the voice guidance in real time according to changes in environmental sound. Thus, by automatically adjusting the volume of the voice guidance according to the surrounding environmental sound, more appropriate guidance can be provided. Some or all of the above-described processing in the provision unit may be performed using AI or may be performed without using AI. For example, the provision unit can input environmental sound data into AI, and the AI can adjust the volume of the voice guidance.

The provision unit can provide voice guidance in multiple languages based on the language settings of the elderly person. For example, the provision unit automatically sets the language of the voice guidance based on the language settings of the elderly person's device. For example, when the elderly person uses multiple languages, the provision unit provides a language switching function. For example, when the elderly person selects a specific language, the provision unit provides voice guidance in that language. Thus, by providing voice guidance in multiple languages, information can be provided beyond language barriers. Some or all of the above-described processing in the provision unit may be performed using AI or may be performed without using AI. For example, the provision unit can input language setting data into AI, and the AI can set the language of the voice guidance.

The detection unit can detect abnormalities by integrating information from multiple sensors. For example, the detection unit integrates heart rate and body temperature data to detect abnormal patterns. For example, the detection unit combines movement data and changes in heart rate to detect the risk of falls. For example, the detection unit analyzes body temperature, heart rate, and movement data in real time to detect abnormalities. Thus, by integrating information from multiple sensors, the accuracy of abnormality detection is improved. Some or all of the above-described processing in the detection unit may be performed using AI or may be performed without using AI. For example, the detection unit can input multiple sensor data into AI, and the AI can detect abnormalities.

The detection unit can improve the accuracy of abnormality detection by referring to the history of past abnormality detections. For example, the detection unit adjusts the abnormality detection algorithm based on the history of past abnormality detections. For example, the detection unit refers to the abnormality detection history and learns patterns to reduce false detections. For example, the detection unit analyzes the history of past abnormality detections to improve the accuracy of abnormality detection. Thus, by referring to the history of past abnormality detections, the accuracy of abnormality detection is improved. Some or all of the above-described processing in the detection unit may be performed using AI or may be performed without using AI. For example, the detection unit can input past abnormality detection history data into AI, and the AI can improve the accuracy of abnormality detection.

The detection unit can learn the lifestyle patterns of the elderly person to improve the accuracy of abnormality detection. For example, the detection unit learns the lifestyle patterns of the elderly person and distinguishes between normal and abnormal movements. For example, the detection unit learns the daily rhythm of the elderly person and detects abnormal patterns at an early stage. For example, the detection unit optimizes the abnormality detection algorithm based on the past behavioral data of the elderly person. Thus, by learning the lifestyle patterns of the elderly person, the accuracy of abnormality detection is improved. Some or all of the above-described processing in the detection unit may be performed using AI or may be performed without using AI. For example, the detection unit can input lifestyle pattern data of the elderly person into AI, and the AI can improve the accuracy of abnormality detection.

The detection unit can detect abnormalities based on environmental information around the elderly person. For example, when the surrounding temperature is high, the detection unit detects the risk of heatstroke. For example, when the surrounding humidity is low, the detection unit detects the risk of dehydration. For example, the detection unit combines environmental information and health data of the elderly person to detect abnormalities. Thus, by considering environmental information, the accuracy of abnormality detection is improved. Some or all of the above-described processing in the detection unit may be performed using AI or may be performed without using AI. For example, the detection unit can input environmental information data into AI, and the AI can detect abnormalities.

The notification unit can perform real-time notification to designated contacts or medical institutions after abnormality detection. For example, immediately after an abnormality is detected, the notification unit sends a real-time notification to the designated contact. For example, when an abnormality is detected, the notification unit notifies the medical institution in real time. For example, when an abnormality is detected, the notification unit sends a real-time notification to family members or caregivers. Thus, after abnormality detection, the notification unit can promptly notify designated contacts or medical institutions. Some or all of the above-described processing in the notification unit may be performed using AI or may be performed without using AI. For example, the notification unit can input abnormality detection data into AI, and the AI can perform real-time notification.

The notification unit can improve the accuracy of notifications by referring to the history of past notifications. For example, the notification unit learns patterns to reduce false notifications based on the history of past notifications. For example, the notification unit refers to the notification history and adjusts so as not to repeat the same notification. For example, the notification unit analyzes the history of past notifications to improve the accuracy of notifications. Thus, by referring to the history of past notifications, the accuracy of notifications is improved. Some or all of the above-described processing in the notification unit may be performed using AI or may be performed without using AI. For example, the notification unit can input past notification history data into AI, and the AI can improve the accuracy of notifications.

The notification unit can select the optimal contact or medical institution in consideration of the current location information of the elderly person. For example, the notification unit notifies the nearest medical institution based on the current location of the elderly person. For example, the notification unit selects the optimal contact based on the location information of the elderly person and sends a notification. For example, when the elderly person is on the move, the notification unit changes the notification destination according to the current location. Thus, the notification unit can select the optimal contact or medical institution based on the current location information of the elderly person. Some or all of the above-described processing in the notification unit may be performed using AI or may be performed without using AI. For example, the notification unit can input location information data into AI, and the AI can select the optimal contact or medical institution.

The notification unit can customize the notification content in consideration of the health status of the elderly person. For example, the notification unit provides appropriate notification content according to the health status of the elderly person. For example, the notification unit provides notifications according to the degree of urgency based on the health data of the elderly person. For example, the notification unit monitors the health status of the elderly person in real time and generates appropriate notification content. Thus, by customizing the notification content according to the health status of the elderly person, more appropriate notifications can be provided. Some or all of the above-described processing in the notification unit may be performed using AI or may be performed without using AI. For example, the notification unit can input health data of the elderly person into AI, and the AI can customize the notification content.

The system according to the embodiment is not limited to the above examples and can be variously modified, for example, as follows.

The recognition unit can improve recognition accuracy by using not only the movement of the user's gaze but also voice input. For example, when the elderly person looks at a specific object and says its name by voice, the recognition unit combines gaze and voice to improve recognition accuracy. Furthermore, when the elderly person moves their gaze while giving instructions by voice, the recognition unit can integrate gaze and voice for recognition. Thus, by using both gaze movement and voice input, recognition accuracy is improved. Some or all of the above-described processing in the recognition unit may be performed using AI or may be performed without using AI. For example, the recognition unit can input gaze data and voice data into AI, and the AI can improve recognition accuracy.

The analysis unit can refer to the history of past voice guidance to generate more appropriate guidance. For example, the analysis unit provides optimal guidance based on the history of voice guidance previously received by the elderly person. Furthermore, the analysis unit can refer to the history of past voice guidance and adjust so as not to repeat the same guidance. Thus, by referring to past history, more appropriate voice guidance can be provided. Some or all of the above-described processing in the analysis unit may be performed using AI or may be performed without using AI. For example, the analysis unit can input past voice guidance history data into AI, and the AI can generate guidance.

The provision unit can provide information not only through voice guidance but also by combining visual guidance. For example, the provision unit displays the procedure for use on a display simultaneously with voice guidance. Furthermore, the provision unit can display visual icons or images together with voice guidance. Thus, by combining visual guidance, understanding of information is deepened. Some or all of the above-described processing in the provision unit may be performed using AI or may be performed without using AI. For example, the provision unit can input data of voice guidance and visual guidance into AI, and the AI can provide the information.

The detection unit can detect abnormalities by integrating information from multiple sensors. For example, the detection unit integrates heart rate and body temperature data to detect abnormal patterns. Furthermore, the detection unit can combine movement data and changes in heart rate to detect the risk of falls. Thus, by integrating information from multiple sensors, the accuracy of abnormality detection is improved. Some or all of the above-described processing in the detection unit may be performed using AI or may be performed without using AI. For example, the detection unit can input multiple sensor data into AI, and the AI can detect abnormalities.

The notification unit can select the optimal contact or medical institution in consideration of the current location information of the elderly person. For example, the notification unit notifies the nearest medical institution based on the current location of the elderly person. Furthermore, the notification unit can select the optimal contact based on the location information of the elderly person and send a notification. Thus, the notification unit can select the optimal contact or medical institution based on the current location information of the elderly person. Some or all of the above-described processing in the notification unit may be performed using AI or may be performed without using AI. For example, the notification unit can input location information data into AI, and the AI can select the optimal contact or medical institution.

The following is a brief explanation of the processing flow of Example 1 of the Embodiment.

Step 1: The recognition unit analyzes eye movements and recognizes objects being viewed by the elderly person. For example, the recognition unit identifies objects being viewed by the elderly person using eye-tracking technology, and when a medicine bottle is viewed, the system recognizes this information and explains the usage or intake method by voice.

Step 2: The analysis unit analyzes information recognized by the recognition unit and generates voice guidance. For example, the analysis unit uses speech synthesis technology to generate the recognized information as voice guidance. Step 3: The provision unit provides the voice guidance generated by the analysis unit. For example, the provision unit provides voice guidance to the elderly person using a speaker.

Step 4: The detection unit detects abnormal movements or conditions of the elderly person using IoT sensor technology. For example, the detection unit monitors heart rate and movements in real time and detects falls or abnormal heart rates.

Step 5: The notification unit notifies designated contacts or medical institutions of abnormalities detected by the detection unit. For example, when a fall is detected, the notification unit automatically sends a notification to family members or medical institutions.

### Example 2 of the Embodiment

The elderly support system according to the embodiment of the present invention is a system that utilizes eye-tracking technology and IoT sensor technology to recognize the daily actions and objects of elderly people and provides various information and advice through voice guidance. This elderly support system recognizes objects and actions being viewed by the elderly person using eye-tracking technology. For example, when a specific medicine or tool is viewed, the system explains its usage or intake method by voice. In addition, IoT sensor technology is introduced to immediately detect abnormal movements or conditions of the elderly person. For example, when a fall or abnormal heart rate is detected, the system has a function to automatically notify designated contacts or medical institutions. This allows elderly people to continue living at home with peace of mind. For example, the system recognizes objects and actions being viewed by the elderly person using eye-tracking technology. At this time, the system analyzes the eye movements in detail and identifies which object is being viewed. For example, when the elderly person looks at a medicine bottle, the system recognizes this information and explains the usage or intake method by voice. This enables the elderly person to use the medicine correctly. Next, IoT sensor technology is introduced to immediately detect abnormal movements or conditions of the elderly person. For example, the system monitors the heart rate and movements of the elderly person in real time using sensors, and immediately notifies when an abnormality is detected. This enables prompt response in emergencies. Furthermore, the system has a function to automatically notify designated contacts or medical institutions. For example, when a fall is detected, a notification is automatically sent to family members or medical institutions. This allows elderly people to continue living safely even when alone. With this mechanism, elderly people can continue living at home with peace of mind. By combining eye-tracking technology and IoT sensor technology, the system ensures the safety of elderly people and supports their daily lives. For example, by explaining the usage of medicine by voice, misuse of medicine is prevented, and by detecting abnormal movements and responding quickly, the risk in emergencies is reduced. Thus, the elderly support system supports the daily lives of elderly people and enables them to continue living at home with peace of mind.

The elderly support system according to the embodiment comprises a recognition unit, an analysis unit, a provision unit, a detection unit, and a notification unit. The recognition unit analyzes eye movements and recognizes objects being viewed by the elderly person. For example, the recognition unit identifies objects being viewed by the elderly person using eye-tracking technology. For example, the recognition unit analyzes the eye movements in detail and identifies which object is being viewed. For example, when the elderly person looks at a medicine bottle, the system recognizes this information and explains the usage or intake method by voice. The analysis unit analyzes information recognized by the recognition unit and generates voice guidance. For example, the analysis unit generates voice guidance based on the information recognized by the recognition unit. For example, the analysis unit uses speech synthesis technology to generate the recognized information as voice guidance. The provision unit provides the voice guidance generated by the analysis unit. For example, the provision unit provides the voice guidance generated by the analysis unit to the elderly person. For example, the provision unit provides voice guidance using a speaker. The detection unit detects abnormal movements or conditions of the elderly person using IoT sensor technology. For example, the detection unit monitors the heart rate and movements of the elderly person in real time using IoT sensor technology and immediately notifies when an abnormality is detected. For example, the detection unit detects falls or abnormal heart rates. The notification unit notifies designated contacts or medical institutions of abnormalities detected by the detection unit. For example, the notification unit automatically notifies designated contacts or medical institutions based on abnormalities detected by the detection unit. For example, when a fall is detected, the notification unit automatically sends a notification to family members or medical institutions. Thus, the elderly support system according to the embodiment supports the daily lives of elderly people and enables them to continue living at home with peace of mind.

The recognition unit can recognize objects being viewed by the elderly person using eye-tracking technology. For example, the recognition unit identifies objects being viewed by the elderly person using eye-tracking technology. For example, the recognition unit analyzes the eye movements in detail and identifies which object is being viewed. For example, when the elderly person looks at a medicine bottle, the system recognizes this information and explains the usage or intake method by voice. By using eye-tracking technology, the recognition unit can accurately recognize objects being viewed by the elderly person. Some or all of the above-described processing in the recognition unit may be performed using AI or may be performed without using AI. For example, the recognition unit can input eye movement data into AI, and the AI can perform object recognition.

The analysis unit can analyze information recognized by the recognition unit and generate voice guidance. For example, the analysis unit generates voice guidance based on the information recognized by the recognition unit. For example, the analysis unit uses speech synthesis technology to generate the recognized information as voice guidance. For example, when the recognition unit recognizes a medicine bottle, the analysis unit generates guidance that explains the usage or intake method by voice. Thus, the analysis unit can analyze recognized information and generate appropriate voice guidance. Some or all of the above-described processing in the analysis unit may be performed using AI or may be performed without using AI. For example, the analysis unit can input information from the recognition unit into AI, and the AI can generate voice guidance.

The provision unit can provide the voice guidance generated by the analysis unit. For example, the provision unit provides the voice guidance generated by the analysis unit to the elderly person. For example, the provision unit provides voice guidance using a speaker. For example, the provision unit provides the voice guidance on the usage or intake method of medicine generated by the analysis unit to the elderly person. Thus, the provision unit can provide the generated voice guidance to the elderly person. Some or all of the above-described processing in the provision unit may be performed using AI or may be performed without using AI. For example, the provision unit can input voice guidance from the analysis unit into AI, and the AI can provide the voice guidance.

The detection unit can detect abnormal movements or conditions of the elderly person using IoT sensor technology. For example, the detection unit monitors the heart rate and movements of the elderly person in real time using IoT sensor technology and immediately notifies when an abnormality is detected. For example, the detection unit detects falls or abnormal heart rates. For example, the detection unit monitors the heart rate and movements of the elderly person in real time using sensors and immediately notifies when an abnormality is detected. Thus, by using IoT sensor technology, the detection unit can accurately detect abnormal movements or conditions of the elderly person. Some or all of the above-described processing in the detection unit may be performed using AI or may be performed without using AI. For example, the detection unit can input data from sensors into AI, and the AI can detect abnormalities.

The notification unit can notify designated contacts or medical institutions of abnormalities detected by the detection unit. For example, the notification unit automatically notifies designated contacts or medical institutions based on abnormalities detected by the detection unit. For example, when a fall is detected, the notification unit automatically sends a notification to family members or medical institutions. For example, when the detection unit detects an abnormal heart rate, the notification unit automatically notifies designated contacts or medical institutions. Thus, the notification unit can promptly notify designated contacts or medical institutions of detected abnormalities. Some or all of the above-described processing in the notification unit may be performed using AI or may be performed without using AI. For example, the notification unit can input abnormal data from the detection unit into AI, and the AI can perform the notification.

The recognition unit can estimate the emotions of the elderly person and adjust recognition accuracy based on the estimated emotions. For example, when the elderly person is nervous, the recognition unit analyzes not only eye movements but also facial expressions to improve recognition accuracy. For example, when the elderly person is relaxed, the recognition unit performs recognition based only on eye movements to reduce processing load. For example, when the elderly person is tired, the recognition unit adjusts recognition accuracy and integrates information from multiple sensors to prevent misrecognition. Thus, by adjusting recognition accuracy according to the emotions of the elderly person, more accurate recognition is possible. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the recognition unit may be performed using AI or may be performed without using AI. For example, the recognition unit can input emotion data of the elderly person into AI, and the AI can adjust recognition accuracy.

The recognition unit can improve recognition accuracy by analyzing not only eye movements but also facial expressions and head movements. For example, the recognition unit analyzes facial expressions in conjunction with eye movements to accurately recognize whether a specific object is being viewed. For example, the recognition unit combines head movements and eye movements to more accurately determine the direction of gaze. For example, the recognition unit analyzes changes in facial expressions in real time and performs corrections to improve recognition accuracy. Thus, by analyzing facial expressions and head movements together, recognition accuracy is improved. Some or all of the above-described processing in the recognition unit may be performed using AI or may be performed without using AI. For example, the recognition unit can input facial expression data and head movement data into AI, and the AI can improve recognition accuracy.

The recognition unit can refer to the usage history of an object when recognizing a specific object to correct the recognition result. For example, when the recognition unit recognizes an object that the elderly person has frequently used in the past, it refers to the usage history to correct the recognition result. For example, when the recognition unit recognizes a specific medicine bottle, it performs accurate recognition based on past usage history. For example, the recognition unit refers to the usage history of tools and corrects the recognition result to prevent misrecognition. Thus, by referring to the usage history, the accuracy of recognition results is improved. Some or all of the above-described processing in the recognition unit may be performed using AI or may be performed without using AI. For example, the recognition unit can input usage history data into AI, and the AI can correct the recognition result.

The recognition unit can estimate the emotions of the elderly person and determine the priority of objects to be recognized based on the estimated emotions. For example, when the elderly person feels anxious, the recognition unit prioritizes the recognition of important objects (such as medicine). For example, when the elderly person is relaxed, the recognition unit prioritizes the recognition of objects used in daily life. For example, when the elderly person is in a hurry, the recognition unit prioritizes the recognition of objects needed immediately. Thus, by determining the priority of objects to be recognized according to the emotions of the elderly person, important objects can be recognized preferentially. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the recognition unit may be performed using AI or may be performed without using AI. For example, the recognition unit can input emotion data of the elderly person into AI, and the AI can determine the priority of objects to be recognized.

The recognition unit can improve recognition accuracy by using not only the movement of the elderly person's gaze but also voice input. For example, when the elderly person looks at a specific object and says its name by voice, the recognition unit combines gaze and voice to improve recognition accuracy. For example, when the elderly person moves their gaze while giving instructions by voice, the recognition unit integrates gaze and voice for recognition. For example, when the elderly person fixes their gaze while asking a question by voice, the recognition unit combines gaze and voice to correct the recognition result. Thus, by using both gaze movement and voice input, recognition accuracy is improved. Some or all of the above-described processing in the recognition unit may be performed using AI or may be performed without using AI. For example, the recognition unit can input gaze data and voice data into AI, and the AI can improve recognition accuracy.

The recognition unit can adjust recognition accuracy based on environmental information around the elderly person. For example, when the lighting is dim, the recognition unit enhances the analysis of eye movements to improve recognition accuracy. For example, when the surrounding noise is loud, the recognition unit prioritizes gaze analysis to reduce the influence of voice input. For example, when the lighting is bright, the recognition unit combines eye movements and facial expressions to improve recognition accuracy. Thus, by considering environmental information, recognition accuracy is improved. Some or all of the above-described processing in the recognition unit may be performed using AI or may be performed without using AI. For example, the recognition unit can input environmental information data into AI, and the AI can adjust recognition accuracy.

The analysis unit can estimate the emotions of the elderly person and adjust the content of the voice guidance based on the estimated emotions. For example, when the elderly person feels anxious, the analysis unit provides voice guidance with content that gives a sense of security. For example, when the elderly person is relaxed, the analysis unit provides voice guidance with detailed explanations. For example, when the elderly person is in a hurry, the analysis unit provides concise and prompt voice guidance. Thus, by adjusting the content of the voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input emotion data of the elderly person into AI, and the AI can adjust the content of the voice guidance.

The analysis unit can analyze information from the recognition unit in real time and generate voice guidance immediately. For example, immediately after the recognition unit recognizes a specific object, the analysis unit immediately explains the usage of the object by voice. For example, when the recognition unit recognizes an action of the elderly person, the analysis unit immediately provides appropriate advice by voice. For example, when the recognition unit detects abnormal movements, the analysis unit immediately generates a warning voice. Thus, by analyzing in real time and generating voice guidance immediately, prompt response is possible. Some or all of the above-described processing in the analysis unit may be performed using AI or may be performed without using AI. For example, the analysis unit can input information from the recognition unit into AI, and the AI can generate voice guidance in real time.

The analysis unit can refer to the history of past voice guidance to generate more appropriate guidance. For example, the analysis unit provides optimal guidance based on the history of voice guidance previously received by the elderly person. For example, the analysis unit refers to the history of past voice guidance and adjusts so as not to repeat the same guidance. For example, the analysis unit analyzes the effectiveness of guidance previously received by the elderly person and generates more effective guidance. Thus, by referring to past history, more appropriate voice guidance can be provided. Some or all of the above-described processing in the analysis unit may be performed using AI or may be performed without using AI. For example, the analysis unit can input past voice guidance history data into AI, and the AI can generate guidance.

The analysis unit can estimate the emotions of the elderly person and adjust the tone and speed of the voice guidance based on the estimated emotions. For example, when the elderly person is nervous, the analysis unit provides voice guidance in a calm tone and at a slow speed. For example, when the elderly person is relaxed, the analysis unit provides voice guidance in a cheerful tone and at a normal speed. For example, when the elderly person is in a hurry, the analysis unit provides quick and concise voice guidance. Thus, by adjusting the tone and speed of the voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input emotion data of the elderly person into AI, and the AI can adjust the tone and speed of the voice guidance.

The analysis unit can generate voice guidance based on the health status of the elderly person. For example, when the heart rate of the elderly person is high, the analysis unit provides advice for relaxation by voice. For example, when the blood pressure of the elderly person is low, the analysis unit provides voice guidance to encourage appropriate actions. For example, the analysis unit monitors the health status of the elderly person in real time and generates appropriate voice guidance. Thus, by considering the health status of the elderly person, more appropriate voice guidance can be provided. Some or all of the above-described processing in the analysis unit may be performed using AI or may be performed without using AI. For example, the analysis unit can input health data of the elderly person into AI, and the AI can generate voice guidance.

The analysis unit can refer to the schedule information of the elderly person to provide voice guidance at an appropriate timing. For example, the analysis unit notifies the medication time by voice based on the schedule of the elderly person. For example, the analysis unit provides advice at an appropriate timing according to the schedule of the elderly person. For example, the analysis unit reminds important appointments by voice based on the schedule information of the elderly person. Thus, by referring to schedule information, voice guidance can be provided at an appropriate timing. Some or all of the above-described processing in the analysis unit may be performed using AI or may be performed without using AI. For example, the analysis unit can input schedule information data into AI, and the AI can determine the timing for providing voice guidance.

The provision unit can estimate the emotions of the elderly person and adjust the method of providing voice guidance based on the estimated emotions. For example, when the elderly person feels anxious, the provision unit provides voice guidance in a tone that gives a sense of security. For example, when the elderly person is relaxed, the provision unit provides voice guidance with detailed explanations. For example, when the elderly person is in a hurry, the provision unit provides concise and prompt voice guidance. Thus, by adjusting the method of providing voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the provision unit may be performed using AI or may be performed without using AI. For example, the provision unit can input emotion data of the elderly person into AI, and the AI can adjust the method of providing voice guidance.

The provision unit can provide information not only through voice guidance but also by combining visual guidance. For example, the provision unit displays the procedure for use on a display simultaneously with voice guidance. For example, the provision unit displays visual icons or images together with voice guidance. For example, the provision unit displays the content of the voice guidance as text on a display for visual confirmation. Thus, by combining visual guidance, understanding of information is deepened. Some or all of the above-described processing in the provision unit may be performed using AI or may be performed without using AI. For example, the provision unit can input data of voice guidance and visual guidance into AI, and the AI can provide the information.

The provision unit can adjust the volume and frequency of the voice guidance in consideration of the hearing condition of the elderly person. For example, the provision unit automatically adjusts the volume of the voice guidance according to the hearing condition of the elderly person. For example, the provision unit adjusts the frequency of the voice guidance based on the hearing condition of the elderly person. For example, the provision unit monitors the hearing condition of the elderly person in real time and provides voice guidance at an appropriate volume and frequency. Thus, by adjusting the volume and frequency of the voice guidance according to the hearing condition of the elderly person, more appropriate guidance can be provided. Some or all of the above-described processing in the provision unit may be performed using AI or may be performed without using AI. For example, the provision unit can input hearing data of the elderly person into AI, and the AI can adjust the volume and frequency of the voice guidance.

The provision unit can estimate the emotions of the elderly person and customize the content of the voice guidance based on the estimated emotions. For example, when the elderly person feels anxious, the provision unit provides voice guidance with content that gives a sense of security. For example, when the elderly person is relaxed, the provision unit provides voice guidance with detailed explanations. For example, when the elderly person is in a hurry, the provision unit provides concise and prompt voice guidance. Thus, by customizing the content of the voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the provision unit may be performed using AI or may be performed without using AI. For example, the provision unit can input emotion data of the elderly person into AI, and the AI can customize the content of the voice guidance.

The provision unit can detect environmental sounds around the elderly person and automatically adjust the volume of the voice guidance. For example, when the surrounding environmental sound is loud, the provision unit automatically increases the volume of the voice guidance. For example, when the surroundings are quiet, the provision unit automatically lowers the volume of the voice guidance. For example, the provision unit adjusts the volume of the voice guidance in real time according to changes in environmental sound. Thus, by automatically adjusting the volume of the voice guidance according to the surrounding environmental sound, more appropriate guidance can be provided. Some or all of the above-described processing in the provision unit may be performed using AI or may be performed without using AI. For example, the provision unit can input environmental sound data into AI, and the AI can adjust the volume of the voice guidance.

The provision unit can provide voice guidance in multiple languages based on the language settings of the elderly person. For example, the provision unit automatically sets the language of the voice guidance based on the language settings of the elderly person's device. For example, when the elderly person uses multiple languages, the provision unit provides a language switching function. For example, when the elderly person selects a specific language, the provision unit provides voice guidance in that language. Thus, by providing voice guidance in multiple languages, information can be provided beyond language barriers. Some or all of the above-described processing in the provision unit may be performed using AI or may be performed without using AI. For example, the provision unit can input language setting data into AI, and the AI can set the language of the voice guidance.

The detection unit can estimate the emotions of the elderly person and adjust the threshold for abnormality detection based on the estimated emotions. For example, when the elderly person feels anxious, the detection unit sets the threshold for abnormality detection lower to detect abnormalities earlier. For example, when the elderly person is relaxed, the detection unit sets the threshold for abnormality detection to normal to prevent false detections. For example, when the elderly person is tired, the detection unit adjusts the threshold for abnormality detection to detect abnormalities at an appropriate timing. Thus, by adjusting the threshold for abnormality detection according to the emotions of the elderly person, more appropriate abnormality detection is possible. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the detection unit may be performed using AI or may be performed without using AI. For example, the detection unit can input emotion data of the elderly person into AI, and the AI can adjust the threshold for abnormality detection.

The detection unit can detect abnormalities by integrating information from multiple sensors. For example, the detection unit integrates heart rate and body temperature data to detect abnormal patterns. For example, the detection unit combines movement data and changes in heart rate to detect the risk of falls. For example, the detection unit analyzes body temperature, heart rate, and movement data in real time to detect abnormalities. Thus, by integrating information from multiple sensors, the accuracy of abnormality detection is improved. Some or all of the above-described processing in the detection unit may be performed using AI or may be performed without using AI. For example, the detection unit can input multiple sensor data into AI, and the AI can detect abnormalities.

The detection unit can improve the accuracy of abnormality detection by referring to the history of past abnormality detections. For example, the detection unit adjusts the abnormality detection algorithm based on the history of past abnormality detections. For example, the detection unit refers to the abnormality detection history and learns patterns to reduce false detections. For example, the detection unit analyzes the history of past abnormality detections to improve the accuracy of abnormality detection. Thus, by referring to the history of past abnormality detections, the accuracy of abnormality detection is improved. Some or all of the above-described processing in the detection unit may be performed using AI or may be performed without using AI. For example, the detection unit can input past abnormality detection history data into AI, and the AI can improve the accuracy of abnormality detection.

The detection unit can estimate the emotions of the elderly person and determine the priority of abnormality detection based on the estimated emotions. For example, when the elderly person feels anxious, the detection unit prioritizes the detection of important abnormalities (e.g., falls). For example, when the elderly person is relaxed, the detection unit prioritizes the detection of daily abnormalities (e.g., minor movement changes). For example, when the elderly person is in a hurry, the detection unit prioritizes the detection of abnormalities that require immediate response. Thus, by determining the priority of abnormality detection according to the emotions of the elderly person, important abnormalities can be detected preferentially. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the detection unit may be performed using AI or may be performed without using AI. For example, the detection unit can input emotion data of the elderly person into AI, and the AI can determine the priority of abnormality detection.

The detection unit can learn the lifestyle patterns of the elderly person to improve the accuracy of abnormality detection. For example, the detection unit learns the lifestyle patterns of the elderly person and distinguishes between normal and abnormal movements. For example, the detection unit learns the daily rhythm of the elderly person and detects abnormal patterns at an early stage. For example, the detection unit optimizes the abnormality detection algorithm based on the past behavioral data of the elderly person. Thus, by learning the lifestyle patterns of the elderly person, the accuracy of abnormality detection is improved. Some or all of the above-described processing in the detection unit may be performed using AI or may be performed without using AI. For example, the detection unit can input lifestyle pattern data of the elderly person into AI, and the AI can improve the accuracy of abnormality detection.

The detection unit can detect abnormalities based on environmental information around the elderly person. For example, when the surrounding temperature is high, the detection unit detects the risk of heatstroke. For example, when the surrounding humidity is low, the detection unit detects the risk of dehydration. For example, the detection unit combines environmental information and health data of the elderly person to detect abnormalities. Thus, by considering environmental information, the accuracy of abnormality detection is improved. Some or all of the above-described processing in the detection unit may be performed using AI or may be performed without using AI. For example, the detection unit can input environmental information data into AI, and the AI can detect abnormalities.

The notification unit can estimate the emotions of the elderly person and adjust the notification content based on the estimated emotions. For example, when the elderly person feels anxious, the notification unit provides notifications with content that gives a sense of security. For example, when the elderly person is relaxed, the notification unit provides notifications with detailed explanations. For example, when the elderly person is in a hurry, the notification unit provides concise and prompt notifications. Thus, by adjusting the notification content according to the emotions of the elderly person, more appropriate notifications can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the notification unit may be performed using AI or may be performed without using AI. For example, the notification unit can input emotion data of the elderly person into AI, and the AI can adjust the notification content.

The notification unit can perform real-time notification to designated contacts or medical institutions after abnormality detection. For example, immediately after an abnormality is detected, the notification unit sends a real-time notification to the designated contact. For example, when an abnormality is detected, the notification unit notifies the medical institution in real time. For example, when an abnormality is detected, the notification unit sends a real-time notification to family members or caregivers. Thus, after abnormality detection, the notification unit can promptly notify designated contacts or medical institutions. Some or all of the above-described processing in the notification unit may be performed using AI or may be performed without using AI. For example, the notification unit can input abnormality detection data into AI, and the AI can perform real-time notification.

The notification unit can improve the accuracy of notifications by referring to the history of past notifications. For example, the notification unit learns patterns to reduce false notifications based on the history of past notifications. For example, the notification unit refers to the notification history and adjusts so as not to repeat the same notification. For example, the notification unit analyzes the history of past notifications to improve the accuracy of notifications. Thus, by referring to the history of past notifications, the accuracy of notifications is improved. Some or all of the above-described processing in the notification unit may be performed using AI or may be performed without using AI. For example, the notification unit can input past notification history data into AI, and the AI can improve the accuracy of notifications.

The notification unit can estimate the emotions of the elderly person and determine the priority of notifications based on the estimated emotions. For example, when the elderly person feels anxious, the notification unit prioritizes important notifications (e.g., notifications to medical institutions). For example, when the elderly person is relaxed, the notification unit prioritizes daily notifications (e.g., notifications to family members). For example, when the elderly person is in a hurry, the notification unit prioritizes notifications that require immediate response. Thus, by determining the priority of notifications according to the emotions of the elderly person, important notifications can be sent preferentially. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the notification unit may be performed using AI or may be performed without using AI. For example, the notification unit can input emotion data of the elderly person into AI, and the AI can determine the priority of notifications.

The notification unit can select the optimal contact or medical institution in consideration of the current location information of the elderly person. For example, the notification unit notifies the nearest medical institution based on the current location of the elderly person. For example, the notification unit selects the optimal contact based on the location information of the elderly person and sends a notification. For example, when the elderly person is on the move, the notification unit changes the notification destination according to the current location. Thus, the notification unit can select the optimal contact or medical institution based on the current location information of the elderly person. Some or all of the above-described processing in the notification unit may be performed using AI or may be performed without using AI. For example, the notification unit can input location information data into AI, and the AI can select the optimal contact or medical institution.

The notification unit can customize the notification content in consideration of the health status of the elderly person. For example, the notification unit provides appropriate notification content according to the health status of the elderly person. For example, the notification unit provides notifications according to the degree of urgency based on the health data of the elderly person. For example, the notification unit monitors the health status of the elderly person in real time and generates appropriate notification content. Thus, by customizing the notification content according to the health status of the elderly person, more appropriate notifications can be provided. Some or all of the above-described processing in the notification unit may be performed using AI or may be performed without using AI. For example, the notification unit can input health data of the elderly person into AI, and the AI can customize the notification content.

The system according to the embodiment is not limited to the above examples and can be variously modified, for example, as follows.

The recognition unit can improve recognition accuracy by using not only the movement of the user's gaze but also voice input. For example, when the elderly person looks at a specific object and says its name by voice, the recognition unit combines gaze and voice to improve recognition accuracy. Furthermore, when the elderly person moves their gaze while giving instructions by voice, the recognition unit can integrate gaze and voice for recognition. Thus, by using both gaze movement and voice input, recognition accuracy is improved. Some or all of the above-described processing in the recognition unit may be performed using AI or may be performed without using AI. For example, the recognition unit can input gaze data and voice data into AI, and the AI can improve recognition accuracy.

The analysis unit can refer to the history of past voice guidance to generate more appropriate guidance. For example, the analysis unit provides optimal guidance based on the history of voice guidance previously received by the elderly person. Furthermore, the analysis unit can refer to the history of past voice guidance and adjust so as not to repeat the same guidance. Thus, by referring to past history, more appropriate voice guidance can be provided. Some or all of the above-described processing in the analysis unit may be performed using AI or may be performed without using AI. For example, the analysis unit can input past voice guidance history data into AI, and the AI can generate guidance.

The provision unit can provide information not only through voice guidance but also by combining visual guidance. For example, the provision unit displays the procedure for use on a display simultaneously with voice guidance. Furthermore, the provision unit can display visual icons or images together with voice guidance. Thus, by combining visual guidance, understanding of information is deepened. Some or all of the above-described processing in the provision unit may be performed using AI or may be performed without using AI. For example, the provision unit can input data of voice guidance and visual guidance into AI, and the AI can provide the information.

The detection unit can detect abnormalities by integrating information from multiple sensors. For example, the detection unit integrates heart rate and body temperature data to detect abnormal patterns. Furthermore, the detection unit can combine movement data and changes in heart rate to detect the risk of falls. Thus, by integrating information from multiple sensors, the accuracy of abnormality detection is improved. Some or all of the above-described processing in the detection unit may be performed using AI or may be performed without using AI. For example, the detection unit can input multiple sensor data into AI, and the AI can detect abnormalities.

The notification unit can select the optimal contact or medical institution in consideration of the current location information of the elderly person. For example, the notification unit notifies the nearest medical institution based on the current location of the elderly person. Furthermore, the notification unit can select the optimal contact based on the location information of the elderly person and send a notification. Thus, the notification unit can select the optimal contact or medical institution based on the current location information of the elderly person. Some or all of the above-described processing in the notification unit may be performed using AI or may be performed without using AI. For example, the notification unit can input location information data into AI, and the AI can select the optimal contact or medical institution.

The recognition unit can estimate the emotions of the elderly person and adjust recognition accuracy based on the estimated emotions. For example, when the elderly person is nervous, the recognition unit analyzes not only eye movements but also facial expressions to improve recognition accuracy. Furthermore, when the elderly person is relaxed, the recognition unit can perform recognition based only on eye movements to reduce processing load. Thus, by adjusting recognition accuracy according to the emotions of the elderly person, more accurate recognition is possible. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the recognition unit may be performed using AI or may be performed without using AI. For example, the recognition unit can input emotion data of the elderly person into AI, and the AI can adjust recognition accuracy.

The analysis unit can estimate the emotions of the elderly person and adjust the content of the voice guidance based on the estimated emotions. For example, when the elderly person feels anxious, the analysis unit provides voice guidance with content that gives a sense of security. Furthermore, when the elderly person is relaxed, the analysis unit can provide voice guidance with detailed explanations. Thus, by adjusting the content of the voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the analysis unit may be performed using AI or may be performed without using AI. For example, the analysis unit can input emotion data of the elderly person into AI, and the AI can adjust the content of the voice guidance.

The provision unit can estimate the emotions of the elderly person and adjust the method of providing voice guidance based on the estimated emotions. For example, when the elderly person feels anxious, the provision unit provides voice guidance in a tone that gives a sense of security. Furthermore, when the elderly person is relaxed, the provision unit can provide voice guidance with detailed explanations. Thus, by adjusting the method of providing voice guidance according to the emotions of the elderly person, more appropriate guidance can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the provision unit may be performed using AI or may be performed without using AI. For example, the provision unit can input emotion data of the elderly person into AI, and the AI can adjust the method of providing voice guidance.

The detection unit can estimate the emotions of the elderly person and adjust the threshold for abnormality detection based on the estimated emotions. For example, when the elderly person feels anxious, the detection unit sets the threshold for abnormality detection lower to detect abnormalities earlier. Furthermore, when the elderly person is relaxed, the detection unit can set the threshold for abnormality detection to normal to prevent false detections. Thus, by adjusting the threshold for abnormality detection according to the emotions of the elderly person, more appropriate abnormality detection is possible. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the detection unit may be performed using AI or may be performed without using AI. For example, the detection unit can input emotion data of the elderly person into AI, and the AI can adjust the threshold for abnormality detection.

The notification unit can estimate the emotions of the elderly person and adjust the notification content based on the estimated emotions. For example, when the elderly person feels anxious, the notification unit provides notifications with content that gives a sense of security. Furthermore, when the elderly person is relaxed, the notification unit can provide notifications with detailed explanations. Thus, by adjusting the notification content according to the emotions of the elderly person, more appropriate notifications can be provided. Emotion estimation is realized, for example, by using an emotion estimation function such as an emotion engine or generative AI. The generative AI may be a text generation AI (e.g., LLM) or a multimodal generative AI, but is not limited to such examples. Some or all of the above-described processing in the notification unit may be performed using AI or may be performed without using AI. For example, the notification unit can input emotion data of the elderly person into AI, and the AI can adjust the notification content.

The following is a brief explanation of the processing flow of Example 2 of the Embodiment.

Step 1: The recognition unit analyzes eye movements and recognizes objects being viewed by the elderly person. For example, the recognition unit identifies objects being viewed by the elderly person using eye-tracking technology, and when a medicine bottle is viewed, the system recognizes this information and explains the usage or intake method by voice.

Step 2: The analysis unit analyzes information recognized by the recognition unit and generates voice guidance. For example, the analysis unit uses speech synthesis technology to generate the recognized information as voice guidance. Step 3: The provision unit provides the voice guidance generated by the analysis unit. For example, the provision unit provides voice guidance to the elderly person using a speaker.

Step 4: The detection unit detects abnormal movements or conditions of the elderly person using IoT sensor technology. For example, the detection unit monitors heart rate and movements in real time and detects falls or abnormal heart rates.

Step 5: The notification unit notifies designated contacts or medical institutions of abnormalities detected by the detection unit. For example, when a fall is detected, the notification unit automatically sends a notification to family members or medical institutions.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-described elements, including the recognition unit, analysis unit, provision unit, detection unit, and notification unit, is implemented, for example, in at least one of the smart device 14 and the data processing apparatus 12. For example, the recognition unit is implemented by the camera 42 and control unit 46A of the smart device 14, analyzes the eye movements in detail, and identifies which object is being viewed. The analysis unit is implemented, for example, by the specific processing unit 290 of the data processing apparatus 12, and generates voice guidance based on the information recognized by the recognition unit. The provision unit provides voice guidance using, for example, the speaker 40B of the smart device 14. The detection unit is implemented, for example, by the IoT sensor and control unit 46A of the smart device 14, and monitors abnormal movements or conditions of the elderly person in real time. The notification unit is implemented, for example, by the specific processing unit 290 of the data processing apparatus 12, and automatically notifies designated contacts or medical institutions based on abnormalities detected by the detection unit. The correspondence between each unit and the device or control unit is not limited to the above examples and can be variously modified.

### Second Embodiment

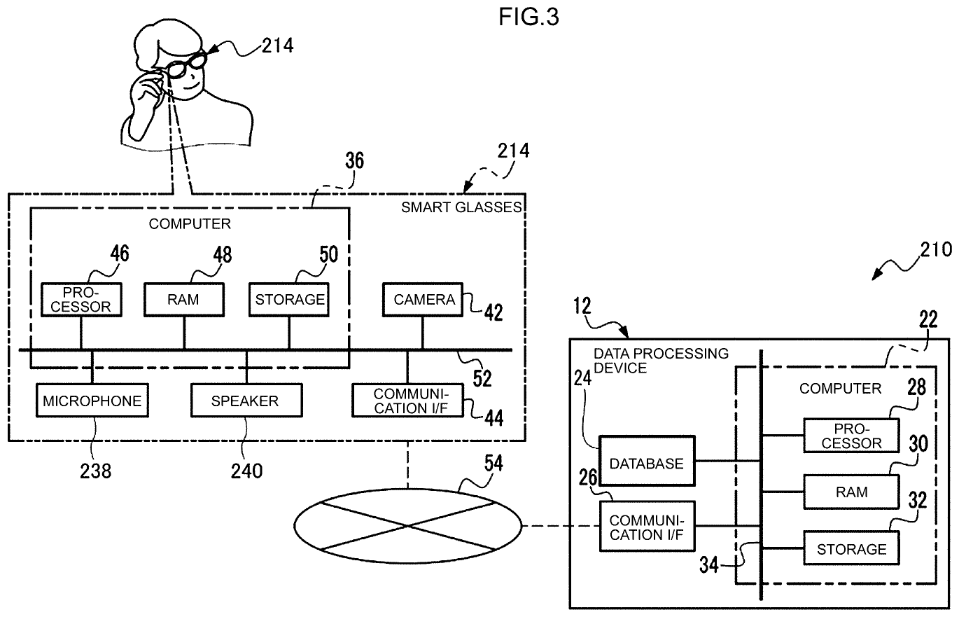

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

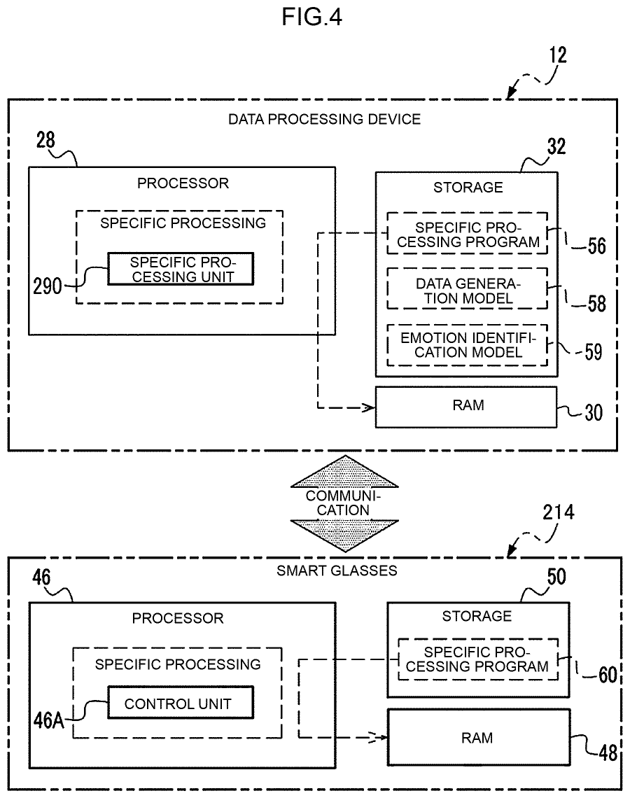

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-described elements, including the recognition unit, analysis unit, provision unit, detection unit, and notification unit, is implemented, for example, in at least one of the smart glasses 214 and the data processing apparatus 12. For example, the recognition unit is implemented by the camera 42 and control unit 46A of the smart glasses 214, analyzes the eye movements in detail, and identifies which object is being viewed. The analysis unit is implemented, for example, by the specific processing unit 290 of the data processing apparatus 12, and generates voice guidance based on the information recognized by the recognition unit. The provision unit provides voice guidance using, for example, the speaker 240 of the smart glasses 214. The detection unit is implemented, for example, by the IoT sensor and control unit 46A of the smart glasses 214, and monitors abnormal movements or conditions of the elderly person in real time. The notification unit is implemented, for example, by the specific processing unit 290 of the data processing apparatus 12, and automatically notifies designated contacts or medical institutions based on abnormalities detected by the detection unit. The correspondence between each unit and the device or control unit is not limited to the above examples and can be variously modified.

### Third Embodiment

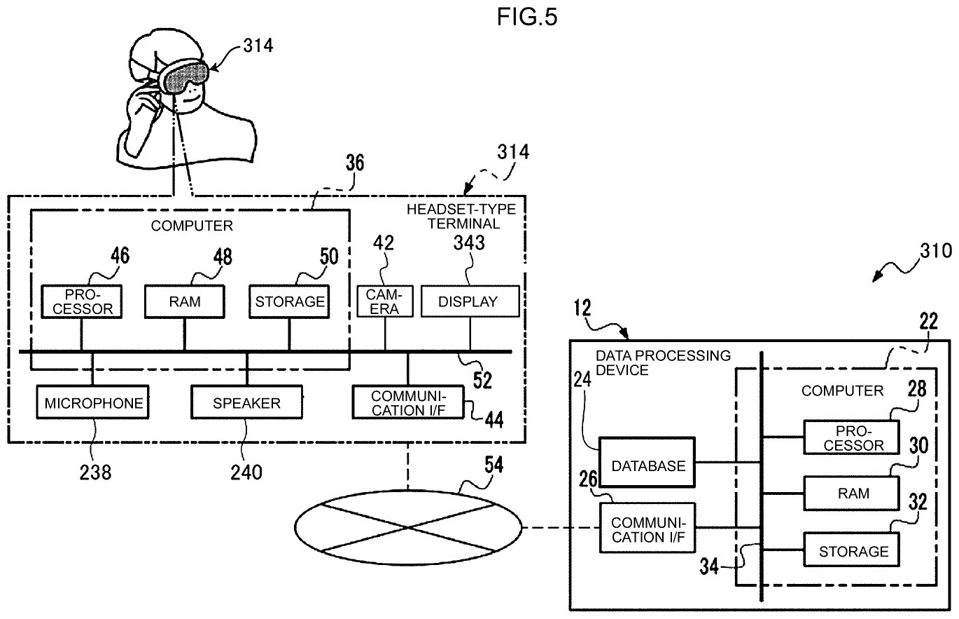

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

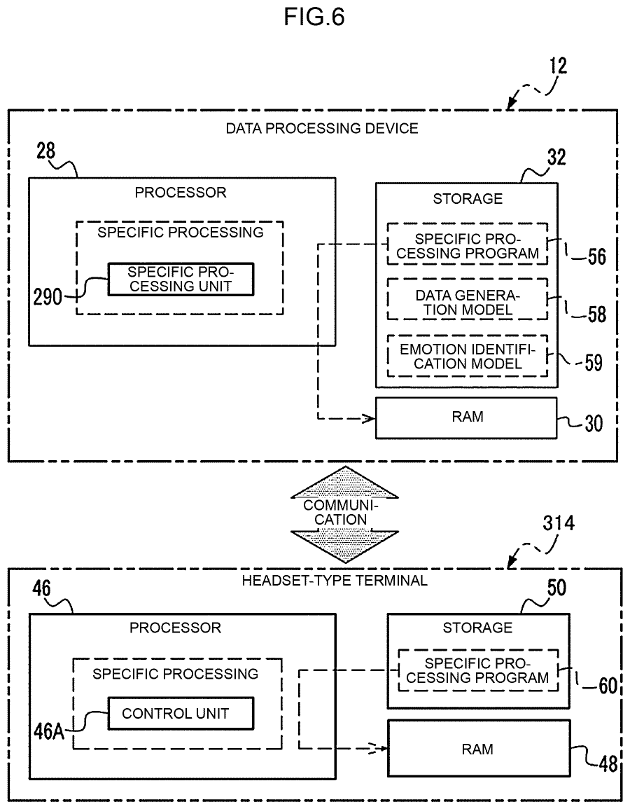

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-described elements, including the recognition unit, analysis unit, provision unit, detection unit, and notification unit, is implemented, for example, in at least one of the headset-type terminal 314 and the data processing apparatus 12. For example, the recognition unit is implemented by the camera 42 and control unit 46A of the headset-type terminal 314, analyzes the eye movements in detail, and identifies which object is being viewed. The analysis unit is implemented, for example, by the specific processing unit 290 of the data processing apparatus 12, and generates voice guidance based on the information recognized by the recognition unit. The provision unit provides voice guidance using, for example, the speaker 240 of the headset-type terminal 314. The detection unit is implemented, for example, by the IoT sensor and control unit 46A of the headset-type terminal 314, and monitors abnormal movements or conditions of the elderly person in real time. The notification unit is implemented, for example, by the specific processing unit 290 of the data processing apparatus 12, and automatically notifies designated contacts or medical institutions based on abnormalities detected by the detection unit. The correspondence between each unit and the device or control unit is not limited to the above examples and can be variously modified.

### Fourth Embodiment

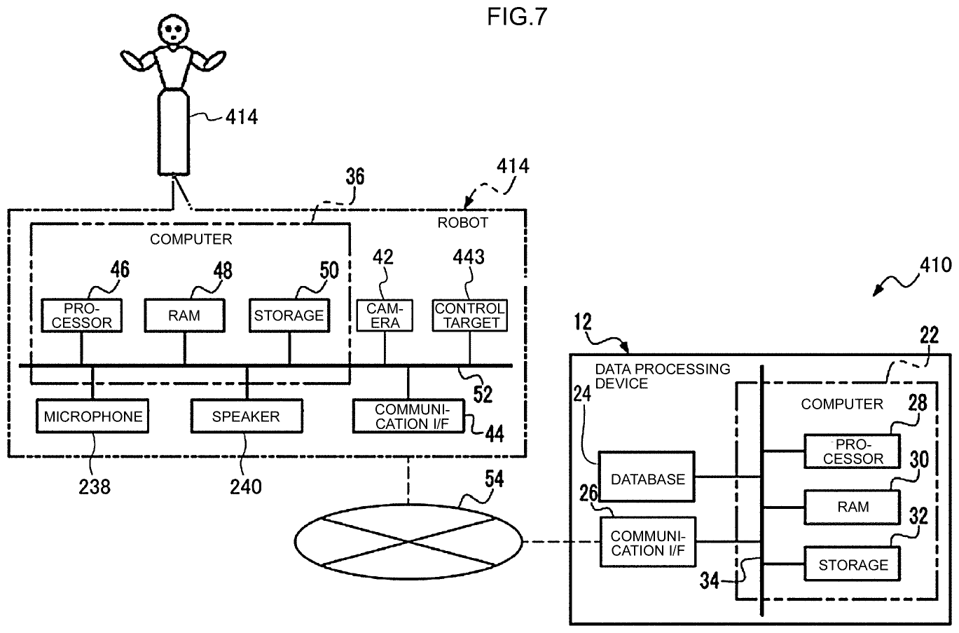

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

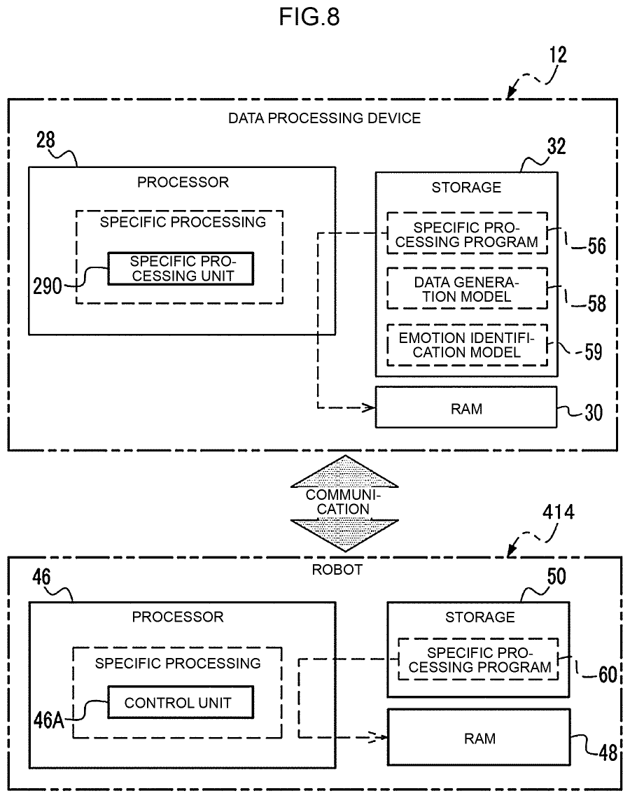

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-described elements, including the recognition unit, analysis unit, provision unit, detection unit, and notification unit, is implemented, for example, in at least one of the robot 414 and the data processing apparatus 12. For example, the recognition unit is implemented by the camera 42 and control unit 46A of the robot 414, analyzes the eye movements in detail, and identifies which object is being viewed. The analysis unit is implemented, for example, by the specific processing unit 290 of the data processing apparatus 12, and generates voice guidance based on the information recognized by the recognition unit. The provision unit provides voice guidance using, for example, the speaker 240 of the robot 414. The detection unit is implemented, for example, by the IoT sensor and control unit 46A of the robot 414, and monitors abnormal movements or conditions of the elderly person in real time. The notification unit is implemented, for example, by the specific processing unit 290 of the data processing apparatus 12, and automatically notifies designated contacts or medical institutions based on abnormalities detected by the detection unit. The correspondence between each unit and the device or control unit is not limited to the above examples and can be variously modified.

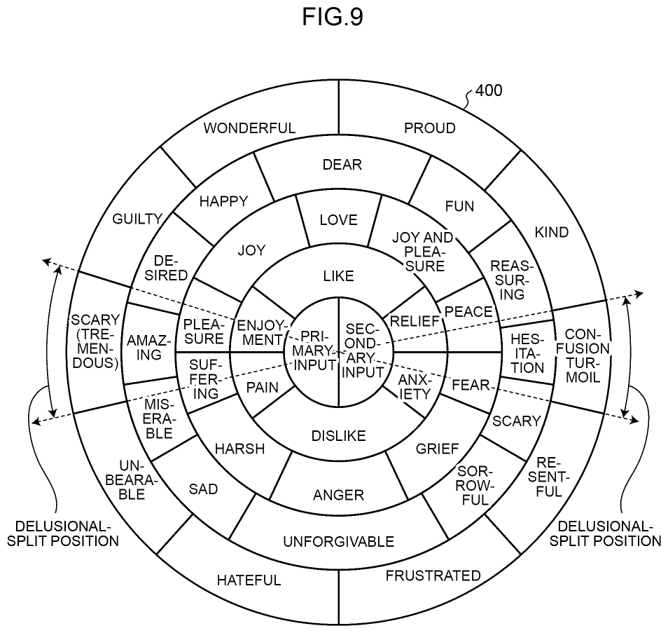

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

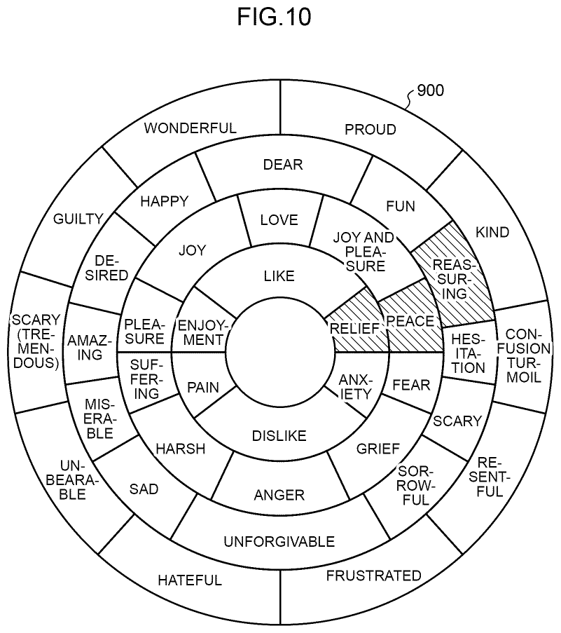

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. Additionally, the examples described above were explained by dividing into form example 1 and form example 2, but these may be combined.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Additional Note 1

A system comprising: a recognition unit that analyzes eye movements and recognizes objects being viewed by an elderly person; an analysis unit that analyzes information recognized by the recognition unit and generates voice guidance; a provision unit that provides the voice guidance generated by the analysis unit; a detection unit that detects abnormal movements or conditions of the elderly person using IoT sensor technology; and a notification unit that notifies designated contacts or medical institutions of abnormalities detected by the detection unit.

### Additional Note 2

The system according to Additional Note 1, wherein the recognition unit recognizes objects being viewed by the elderly person using eye-tracking technology.

### Additional Note 3

The system according to Additional Note 1, wherein the analysis unit analyzes information recognized by the recognition unit and generates voice guidance.

### Additional Note 4

The system according to Additional Note 1, wherein the provision unit provides the voice guidance generated by the analysis unit.

### Additional Note 5

The system according to Additional Note 1, wherein the detection unit detects abnormal movements or conditions of the elderly person using IoT sensor technology.

### Additional Note 6

The system according to Additional Note 1, wherein the notification unit notifies designated contacts or medical institutions of abnormalities detected by the detection unit.

### Additional Note 7

The system according to Additional Note 2, wherein the recognition unit estimates the emotions of the elderly person and adjusts recognition accuracy based on the estimated emotions.

### Additional Note 8

The system according to Additional Note 2, wherein the recognition unit analyzes not only eye movements but also facial expressions and head movements to improve recognition accuracy.

### Additional Note 9

The system according to Additional Note 2, wherein the recognition unit, when recognizing a specific object, refers to the usage history of the object to correct the recognition result.

### Additional Note 10

The system according to Additional Note 2, wherein the recognition unit estimates the emotions of the elderly person and determines the priority of objects to be recognized based on the estimated emotions.

### Additional Note 11

The system according to Additional Note 2, wherein the recognition unit improves recognition accuracy by using not only the movement of the elderly person's gaze but also voice input.

### Additional Note 12

The system according to Additional Note 2, wherein the recognition unit adjusts recognition accuracy based on environmental information around the elderly person.

### Additional Note 13

The system according to Additional Note 3, wherein the analysis unit estimates the emotions of the elderly person and adjusts the content of the voice guidance based on the estimated emotions.

### Additional Note 14

The system according to Additional Note 3, wherein the analysis unit analyzes information from the recognition unit in real time and generates voice guidance immediately.

### Additional Note 15

The system according to Additional Note 3, wherein the analysis unit refers to the history of past voice guidance to generate more appropriate guidance.

### Additional Note 16

The system according to Additional Note 3, wherein the analysis unit estimates the emotions of the elderly person and adjusts the tone and speed of the voice guidance based on the estimated emotions.

### Additional Note 17

The system according to Additional Note 3, wherein the analysis unit generates voice guidance based on the health status of the elderly person.

### Additional Note 18

The system according to Additional Note 3, wherein the analysis unit refers to the schedule information of the elderly person to provide voice guidance at an appropriate timing.

### Additional Note 19

The system according to Additional Note 4, wherein the provision unit estimates the emotions of the elderly person and adjusts the method of providing voice guidance based on the estimated emotions.

### Additional Note 20

The system according to Additional Note 4, wherein the provision unit provides information not only through voice guidance but also by combining visual guidance.

### Additional Note 21

The system according to Additional Note 4, wherein the provision unit adjusts the volume and frequency of the voice guidance in consideration of the hearing condition of the elderly person.

### Additional Note 22

The system according to Additional Note 4, wherein the provision unit estimates the emotions of the elderly person and customizes the content of the voice guidance based on the estimated emotions.

### Additional Note 23

The system according to Additional Note 4, wherein the provision unit detects environmental sounds around the elderly person and automatically adjusts the volume of the voice guidance.

### Additional Note 24

The system according to Additional Note 4, wherein the provision unit provides voice guidance in multiple languages based on the language settings of the elderly person.

### Additional Note 25

The system according to Additional Note 5, wherein the detection unit estimates the emotions of the elderly person and adjusts the threshold for abnormality detection based on the estimated emotions.

### Additional Note 26

The system according to Additional Note 5, wherein the detection unit integrates information from multiple sensors to detect abnormalities.

### Additional Note 27

The system according to Additional Note 5, wherein the detection unit refers to the history of past abnormality detections to improve the accuracy of abnormality detection.

### Additional Note 28

The system according to Additional Note 5, wherein the detection unit estimates the emotions of the elderly person and determines the priority of abnormality detection based on the estimated emotions.

### Additional Note 29

The system according to Additional Note 5, wherein the detection unit learns the lifestyle patterns of the elderly person to improve the accuracy of abnormality detection.

### Additional Note 30

The system according to Additional Note 5, wherein the detection unit detects abnormalities based on environmental information around the elderly person.

### Additional Note 31

The system according to Additional Note 6, wherein the notification unit estimates the emotions of the elderly person and adjusts the notification content based on the estimated emotions.

### Additional Note 32

The system according to Additional Note 6, wherein the notification unit performs real-time notification to designated contacts or medical institutions after abnormality detection.

### Additional Note 33

The system according to Additional Note 6, wherein the notification unit refers to the history of past notifications to improve the accuracy of notifications.

### Additional Note 34

The system according to Additional Note 6, wherein the notification unit estimates the emotions of the elderly person and determines the priority of notifications based on the estimated emotions.

### Additional Note 35

The system according to Additional Note 6, wherein the notification unit selects the optimal contact or medical institution in consideration of the current location information of the elderly person.

### Additional Note 36

The system according to Additional Note 6, wherein the notification unit customizes the notification content in consideration of the health status of the elderly person.

What is claimed is:

1. A system comprising: a recognition unit that analyzes eye movements and recognizes objects being viewed by an elderly person; an analysis unit that analyzes information recognized by the recognition unit and generates voice guidance; a provision unit that provides the voice guidance generated by the analysis unit; a detection unit that detects abnormal movements or conditions of the elderly person using IoT sensor technology; and a notification unit that notifies designated contacts or medical institutions of abnormalities detected by the detection unit.

2. The system according to claim 1, wherein the recognition unit recognizes objects being viewed by the elderly person using eye-tracking technology.

3. The system according to claim 1, wherein the analysis unit analyzes information recognized by the recognition unit and generates voice guidance.

4. The system according to claim 1, wherein the provision unit provides the voice guidance generated by the analysis unit.

5. The system according to claim 1, wherein the detection unit detects abnormal movements or conditions of the elderly person using IoT sensor technology.

6. The system according to claim 1, wherein the notification unit notifies designated contacts or medical institutions of abnormalities detected by the detection unit.

7. The system according to claim 2, wherein the recognition unit estimates the emotions of the elderly person and adjusts recognition accuracy based on the estimated emotions.

8. The system according to claim 2, wherein the recognition unit analyzes not only eye movements but also facial expressions and head movements to improve recognition accuracy.
